(19) United States
(12) Patent Application Publication
(10) Pub. No.: US 2010/0228287 A1
(43) Pub. Date: Sep. 9, 2010

(54) DEVICE FOR ADHERING TO THE SKIN OF A PATIENT

(75) Inventors: Johannes Jeekel, Rotterdam (NL); Henri Batterink, Hoogland (NL)

Correspondence Address: HOFFMANN & BARON, LLP, 6900 JERICHO TURNPIKE, SYOSSET, NY, 11791, US

(73) Assignee: EUROTAPE B.V., Soest (NL)

(21) Appl. No.: 12/682,911

(22) PCT Filed: Oct. 16, 2007
(86) PCT No.: PCT/NL07/00262

§ 371 Date: Apr. 14, 2010

(51) Int. Cl. A61B 17/03

(52) U.S. Cl. 606/215

(57) ABSTRACT

The present invention relates to a device for adhering to the skin of a patient, suitable to make a skin incision there through, as well as to the use of such a device for allowing an incision or excision wound to be made through the said device, and for subsequent closing the wound. In particular, the device is suitable for adhering to the skin at the location the incision or excision is to be made, where after the incision or excision is made through the device in the skin at the envisaged location. The device comprises one or more basic layers (1, 2) that do not abut one another at the location of the wound. In contrast, an open skin area (3) is defined by the basic layer(s), which area is covered by an adhesive surface of a covering sheet (4). Further, adhesive strips (13, 23) are provided that are suitable to be adhered to the skin at both sides of the incision, therewith closing the skin in a highly controlled manner, and enabling excision wounds to be conveniently closed as well

### CROSS-REFERENCE TO RELATED APPLICATIONS

This application is the National Stage of International Application No. PCT/NL2007/000262, which designates the U.S., filed Oct. 16, 2007, the contents of which is incorporated by reference herein.

### FIELD OF THE INVENTION

The present invention relates to a device for adhering to the skin of a patient, suitable to make a skin incision there through, the device comprising means for closure of the incision wound, as well as to the use of such a device for allowing an incision or excision wound to be made in skin through the said device, and for subsequent closing the said wound. The said wound can also be a larger surgical wound. In particular, the device is suitable for adhering to the skin at the location the incision or excision is to be made, where after the incision or excision is made through the device in the skin at the envisaged location.

### BACKGROUND OF THE INVENTION

Such devices are known in the art. E.g. WO 88/08690 describes a wound closure device having a backing layer having an adhesive surface for adhering to the skin and comprising, on the opposed surface, adhesive coated strips that are arranged in an S or Z configuration. These strips are arranged in two parallel arrays, which arrays are distanced from one and another. The incision is made through the baking layer between both arrays, after which the skin can be closed by folding the strips and adhere these onto the backing layer, extending over the location of the incision. The device according to WO 88/08690 may provide for complete coverage of the incision by the strips, which may result in equal closing forces along the edges of the wound, which is a significant improvement over wound closure by stitching. Stitching namely results in high closing forces at the site of the stitches, however significant lower closure forces between the stitches are achieved. The device according to WO 88/08690 has an important disadvantage in that the backing layer remains adhered to the skin, also at the site of the skin edges of the incision. The edges of the incision wound are therefore also covered by the backing layer. The wound edges are brought together by abutting the edges of the incised backing layer to one another by using the strips. There is no direct control of the skin below the backing layer, which may result in suboptimal closing and in suboptimal scar formation.

Such a device is further not suitable for performing excisions from the skin, e.g. when a small melanoma has to be excised. In that case, the excision would also be made in the basic layer, and re-abutting the edges of the basic layer is not well possible, as the excised portion of the layer is missing. The excision wound cannot be closed conveniently.

The same is true in the devices, as described in U.S. Pat. No. 5,336,219, U.S. Pat. No. 6,007,564 and EP 1.675.521. These devices all have a basic layer as described above, where through the incision is to be made.

The same problem is encountered in the device of U.S. Pat. No. 4,531,521, wherein two separate backing layers, which may be held together by a removable cover layer, is placed onto the skin, wherein those layers abut one another. The incision is made precisely between these backing layers. After the incision is made, the wound can be closed by bringing the backing layers back together again. Again, direct contact with the skin surrounding the wound is not possible. Control of wound closing is only by re-abutting the backing layers to one another.

U.S. Pat. No. 4,222,383 describes a closing device, comprising a backing layer to be adhered to the skin, which backing layer comprises two arrays of closing strips, which arrays are arranged at a distance and parallel to one another. Between these arrays, the backing layer is however not adhered to the skin. At this location the incision is to be made, where after the wound is closed by connecting opposingly arranged thin closing strips to one another. These strips are not adhered to the skin or to the basic layer. Although the device according to U.S. Pat. No. 4,222,383 may avoid stitching or stapling the wound, the closing effect will be similar of that of stitching as in each of the arrays, the closing strips are located at a distance from one another. As the wound is only closed at the location of the strips, a high local closing force is obtained at the location of the connected strips, whereas the closing force is significantly lower between the strips.

US2004/0204740 and WO03/053296 describe devices for closing skin wounds without sutures, comprising two separate elongated base strips, each comprising bridging elements. The base strips are to be adhered to the skin adjacent to the skin wound after the incision is made. Then, base strips are connected to one another, therewith closing the wound, by adhering the bridging elements of the one base strip to the other base strip. These devices are not suitable to make an incision there through, and the bridging elements are not suitable to adhere to the skin, only to the opposing base strip, so that the base strips must be located at the edges of the incision wound for skin closure.

### SUMMARY OF THE INVENTION

The device according to the present invention envisages to improve at least one of the above disadvantages, and this is achieved by the device according to the present invention, in particular by the provision of one or more basic layers that do not abut one another at the location of the wound. In contrast, an open skin area is defined by the basic layer(s), which area is covered by an adhesive surface of a covering sheet. Further, adhesive strips are provided that are suitable to be adhered to the skin at both sides of the incision, therewith closing the skin in a highly controlled manner. The device allows an excision or incision to be made in a convenient manner, and enabling excision wounds to be conveniently closed as well.

In a first aspect, the invention provides a device according to the main claim. Further embodiments are described in the dependent claims.

In a second aspect, the invention provides the use of the device according to the invention for allowing an incision or excision wound to be made therethrough in the skin and for closing the skin according to claim 28. Further embodiments are given in claims dependent thereon.

In a third aspect, the invention relates to a device according to claim 34, of which further embodiments are given in claims, dependent thereon.

### BRIEF DESCRIPTION OF THE DRAWINGS

The invention will now be further illustrated by way of example only, by reference to the following figures, which are however in no sense intended to limit the scope of the appended claims.

FIGS. 1 and 2A show a first and a more preferred second embodiment of the device according to the invention respectively, before assembly of cover layer, sheet members, bridging members, bridge covering members and sheets of backing material,

FIG. 2B shows the assembly of the components of FIG. 2A,

FIG. 3 is a top view of an assembly of a first and second sheet member, spacing members and bridging members, made of one piece, wherein the bridging members are folded backward onto the respective sheet members,

FIG. 4 is an overview of the assembly of FIG. 3, wherein the bridging members are each covered by a bridge covering member,

FIG. 5 is an upper view on an assembly as in FIG. 3, wherein the bridging members are closed,

FIG. 6 is a cross section through line X of FIG. 4,

FIG. 7 is a cross section of FIG. 6, wherein the assembly now comprises backing paper and a covering sheet,

FIG. 8 is a cross section of another embodiment of an assembly of a bridging member, sheet member and bridge covering member,

FIG. 9 is a cross section of an arrangement of 2 backing papers for the device according to the present invention,

FIG. 10 is an arrangement of an assembly of 3 backing papers for the device according to the present invention,

FIG. 11 is a schematic overview of different embodiments of the backing paper,

FIG. 12 is an overview of different shapes of bridging members,

FIG. 13 is an overview of different sheet members,

FIG. 14 is an overview of different embodiments of different arrangements of bridging members and bridge covering members,

FIG. 15 shows different embodiments of covering sheets,

FIG. 16 shows different embodiments of alignment markers,

FIGS. 17A-R shows a cartoon of the use of the device according to the present invention for making and closing an incision wound in skin.

### DETAILED DESCRIPTION OF PREFERRED EMBODIMENTS

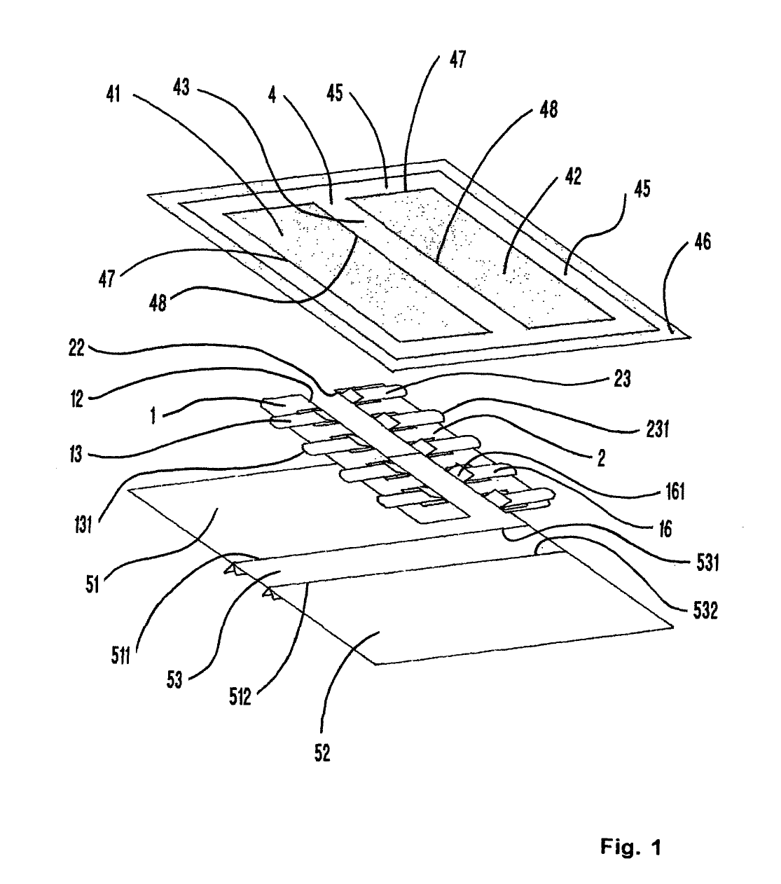

In FIG. 1, a first sheet member 1 and a second sheet member 2 each having a first surface (facing down), and a second surface (facing up) are positioned opposite to one another. The first sheet member 1 has a first opposing edge 12, whereas the second sheet member 2 has a second opposing edge 22. The first and second opposing edges oppose one another, therewith defining a bridge distance there between. The bridge distance is preferably at least 2 cm, leaving about 1 cm of surrounding skin of the wound, more preferably at least 3 cm, leaving about 1.5 cm of surrounding skin of the wound. Preferably, the said bridging distance does not exceed 10 cm, leaving about 5 cm of surrounding skin of the wound, more preferably, the said bridging distance does not exceed 8 cm, leaving about 4 cm of surrounding skin of the wound. In the present case, both first and second opposing edges 12, 22 are straight and run parallel to one another. However, these edges can also be curved, so that the bridging distance can vary accordingly. However, straight opposing edges are preferred. The first surface of both the first and second sheet members 1, 2 comprise a first adhesive which is suitable to adhere the respective sheet member to the skin. The skilled person will be aware of suitable materials for both the adhesive and the sheet members. Preferably, the sheet member is air permeable, and is preferably a polyurethane foil having a thickness, preferably varying from 20-60 μm, more preferably of 25-50 μm, most preferably about 35 μm. With ‘about’ is meant that the thickness can vary 3 μm or less from the said value, i.e. being between 32 and 38 μm. The first adhesive should preferably ensure a good adherence over at least ten days, should preferably be removable without damaging the skin and should preferably also have sufficient tack upon contact with the skin. Preferred examples of such adhesives are hotmelt adhesives based on polyolefine, as are known to the skilled person, who will be aware of suitable alternatives. The length of opposing edges 12 and 22 define, together with the bridging distance there between, an open skin area, in which area the incision in the skin, or excision from the skin, should take place, once the sheet members are placed on the envisaged location on the skin.

The first and second sheet members 1, 2 comprise at their first and second opposing edges 12, 22, respectively, a plurality of bridging members 13, 23, four on the first sheet member 1 and five on the second sheet member 2. It is to be noted that it is preferred to design both first and second sheet members 1,2 in an identical fashion, i.e. having the same number of bridging members 13, 23. Each bridging member 13, 23 comprises a strip of sheet material, having a first and second surface, and the bridging members are folded backward to the first and second sheet members respectively. The sheet material of the bridging members have a first surface facing up when the bridging members are folded backward, and a second surface facing down when the bridging members are folded backward, like in FIG. 1. The first surface comprises a second adhesive, which is suitable to adhere the respective bridging member to the skin upon unfolding, and optionally also to the second surface to the opposing sheet member. Again this sheet material is preferably air permeable, and preferably a polyurethane foil, more preferably of the above identified specifications.

On top of the sheet members, a covering sheet 4 is positioned, comprising a first part 41, covering the first sheet member 1, a second part 42, covering the second sheet member 2, and a third part 43 covering the open skin area 3. Further, the covering sheet comprises in the embodiment shown, a circumfering portion 45, circumfering the first, second and third portions of the covering sheet. The covering sheet also comprises a first surface, facing down to the second surface of the first and second sheet members and to the open skin area, and a second surface, facing up. The first surface of the covering sheet 4 comprises a third adhesive at the third portion 43, and a fourth adhesive at he circumfering portion 45. Said third and fourth adhesive are also suitable to be releasably adhered to the skin, and are preferably the same adhesive. The cover sheet preferably comprises an additional edge portion 46 along the perimeter of the circumfering portion 45. Said additional edge portion can be made of a sheet material of more stiffness and/or strength than the rest of the covering sheet to confer additional strength to the device to improve the handling properties thereof. A suitable material is e.g. polypropylene, whereas the preferred material for the sheet member is polyurethane foil. Said additional edge portion can also e.g. be of a different colour than the rest of the covering sheet, to improve handling of the device.

The covering sheet preferably comprises first and/or second visual indication means 47 and 48, respectively, e.g. in the form of a coloured line. The first visual indication means indicate the location of the first and second sheet members, whereas the second visual indication means indicate the location of the open skin access area. In the present embodiment both first and second visual indication means are combined in the form of a box. These visual indication means can have any form, as long as the respective locations are indicated therewith. For example, the box, enclosed by the lines 47, 48 can be hatched. The visual indication means help the medical staff in positioning the device onto the skin of the patient. Also, the physician will know where to make the incision without the risk of cutting through the sheet members.

Upon placing the device according to the invention on the skin of the patient, the device will adhere to the skin by the adhering first and second sheet members, the third portion of the sheet member and, if present, by the circumfering portion of the covering sheet. The covering sheet covers the sheet members and the bridging members located thereon.

Preferably, the covering sheet does not adhere to the sheet members, as the covering sheet is to be removed from the skin after the incision of excision is made, whereas the sheet members should remain adhered to the skin with the bridging members in back-folded position. To this end, the first surface of the covering sheet 4 is, covering the first and second sheet members 1,2 (i.e. at the first and second portion 41, 42 of the covering sheet 4) preferably free of adhesive. However, the adhesive of the covering sheet 4 may, to this end, locally be shielded, e.g. by a piece of sheet material covering the adhesive. If the covering sheet would adhere to the sheet members and/or the bridging members, the sheet members could also release from the skin, or the bridging members could move from their back-folded position, or even be damaged upon removal of the covering sheet. However, the adhesive can be chosen such that the tack between the covering sheet and the sheet members and the bridging members is less than the tack of the sheet member and the skin. And in that case, also the bridging members should, in back folded position, adhere to the respective sheet member, which can be accomplished by providing a suitable fifth adhesive between the second surface of the bridging member and the second surface of the sheet member, at a location where these surfaces abut one another. The tack between these surfaces should again be more than the tack of the third adhesive of the covering sheet in order to ensure proper positioning of the bridging members upon removal of the covering sheet. However, the fifth adhesive must still allow manual release of the bridging member from the respective sheet member.

The first and second sheet members 1, 2 are positioned on sheets of backing material. The sheet of backing material comprises a first, second and third sheet of backing paper 51, 52 and 53 respectively. A suitable material for sheets of backing material is known to the skilled person. The material can e.g. be paper, preferably comprising a silicon coating, but any suitable sheet like backing material may be used. However, a silicon coated material, such as silicon paper, is preferred. In the assembled situation, the backing papers 51, 52 and 53 are releasably attached to the first surface of the first and second sheet members 1, 2. It is also releasably attached to the third portion 43 of the first surface of the covering sheet 4, which covers the open skin area. Also the edges 45 of the covering sheet 4 attach releasably to the backing paper. In the present case, the covering sheet comprises an additional edge of a more rigid material, e.g of polypropylene, to facilitate proper handling of the assembly and removal of the covering sheet. However, any suitable material can be used. The backing paper 51 comprises a first abutting edge 511, abutting a primary abutting edge 531 of the third sheet 53, whereas the second sheet 52 comprises a second abutting edge 512, abutting the third sheet via the secondary abutting edge 532 of the third sheet 53. The sheets 51, 52 and 53 all extend with a certain length beyond their abutting edges 511, 512, 531 and 532 respectively, in order to facilitate removal thereof

Once assembled, the device according to the invention is preferably packed in a suitable sterilisation bag so that the device can be sterilized after production and/or before use.

In a simpler embodiment, the device comprises two sheets of backing material, wherein the third sheet is not present. In that case, abutting edge 511 of the first sheet 51 abuts the second abutting edge 512 of the second sheet 52. The abutting edges of the first, second and third sheets of backing paper preferably run parallel to one another and preferably perpendicular to the length axis of the open skin area, which in this case runs parallel to the first and second opposing edges 11 and 12 of the first and second sheet member.

In the embodiment shown in FIG. 1, the first and second sheet members 1, 2 are not connected to one another, so that the distance there between can be freely chosen.

It is of importance to keep both sheet members in the envisaged position upon removal of the backing paper, in particular, when there is no tack between the covering sheet and the first and second sheet members. In order to provide proper positioning of both sheet members, the backing paper should be removed carefully, to avoid undesired repositioning of the sheet members. By the use of multiple backing papers, this problem can be minimized, as the sheet members can remain properly positioned when only one of the two or three backing papers is removed. As will be shown below, the embodiment having three backing papers is preferred.

The bridging members are provided, on their first surface, with a bridge covering member 16, said bridge covering member having a first surface (facing down) covering the adhesive first surface of the bridging members. The bridge covering members 16 are releasably attached to the respective bridging members 12, 22 by the second adhesive of the bridging members. The bridge covering members 16 are of a suitable sheet like material, keeping the adhesive of the bridging member intact upon the removal of the bridge covering member 16. A suitable material for the bridge covering member 16 is e.g. silicon paper, but the skilled person is aware of any suitable material. Although not shown in the figure, protruding ends 131, 231 of first and second bridging members 13, 23 respectively, can be connected to the respective bridge covering member 16, which is folded backward on the first surface of the bridging member. In the present case, the length of the bridge covering member exceeds that of the respective bridging member, wherein the exceeding end 161 of the bridge covering member 16 is folded backward at the location of the opposing edge of the sheet member that comprises the said bridging member. By this, the exceeding end 161 of the bridge covering member 16 allows being held by hand, in order to pull the bridge covering member 16 from the bridging member 13, 23 and therewith lifting and unfolding the bridging member as will be shown below. It is important that the adhesive of the bridging member 13, 23 allows tearing off the bridge covering member 16, however it should have sufficient tack to remain secured on the skin, as explained above.

In a particular attractive embodiment (not shown) the exceeding end 161 of the bridge covering member extends beyond the end of the bridging member 131, 231 to facilitate be picked up by hand even more. In that case, the length of the bridge covering member exceeds twice that of the respective bridging member.

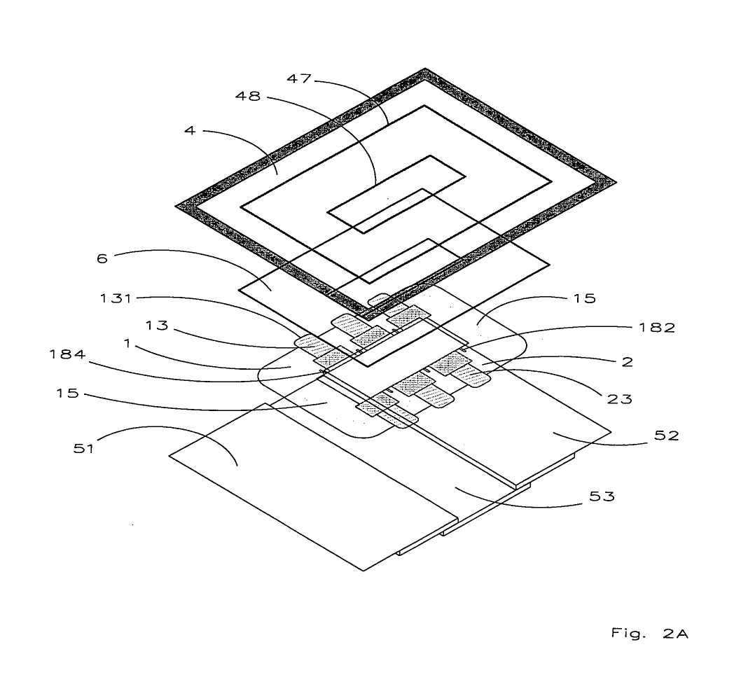

In FIG. 2A, a similar device as that of FIG. 1 is shown with a few additional advantages. Features that are the same with regard to the device as explained in FIG. 1, have the same reference numbers as in FIG. 1, or are not indicated. First of all, the first and second sheet members 1 and 2 are connected to one another by spacing members 15, which are in this case, of the same sheet material of sheet members 1 and 2. Although the spacing members can be made of separate pieces of material and be of different material than the sheet members, the sheet members and spacing members are preferably made of a single piece of sheet material. The spacing members also have a first surface, facing down, and a second surface, facing up. The first surface comprising an adhesive, suitable for releasably adhering the spacing member to the skin. In the present case, the first surface of the first and second sheet members and the first surface of the spacing members are of the same material and preferably, the adhesive of the first sheet members and of the spacing members are identical. The bridging members 13 and 23 are folded backwards onto the first and second sheet members 1, 2 respectively so that the complete second surface of the bridging member faces the second surface of the respective sheet member. However, the sheet member and the bridging member can be dimensioned such, that the protruding end 131 of the bridging member extends beyond the respective sheet member, so that only a portion of the second surface of the bridging member faces the second surface of the respective sheet member. Although the bridging members can be made of separate material and be connected to the first and second sheet members by the aid of a suitable adhesive, the first and second bridging members are preferably an integral part of the first and second sheet members 1, 2 respectively, so that the first sheet member and the first bridging members are made of a single piece of sheet and the second sheet member and bridging members as well. In the present case, both sheet members, the spacing members as well as the bridging members are made of a single piece of sheet material. The sheet members comprise alignment markers 182 and 184 on the second and first sheet member 2, 1 respectively. In the same axis of these alignment markers, a corresponding alignment marker is provided on the bridging members visible from the second surface thereof As the bridging members are in a back folded position, the first surface of the bridging member faces up, which members are also covered by bridge covering members, as indicated in FIG. 1.

In this embodiment, the covering sheet 4 comprises first visual indication means 47, indicating the location of the first and second sheet members 1, 2 and spacing members 15, as well as second visual indication means 48, indicating the location of the open skin access area 3. These means can be designed e.g. as coloured lined in this embodiment. Again, these visual indication means help proper positioning of the device on the skin of the patient, and give a clear indication of the location of the open skin access area, wherein the incision is to be made. Again, any suitable visual indication means can be used, who are known to the skilled person. Coloured lines can be used, or the envisaged areas can be coloured completely, or hatched, etc. Further, in the assembly of this embodiment, the covering sheet adhered to a shielding member 6. By the presence of the shielding member, the first surface of the sheet member can be completely provided with adhesive, and at the location covering the first and second sheet members and the bridging members, the adhesive is shielded by the shielding member, avoiding undesired adherence of the covering sheet with the sheet members and bridging members.

The advantage of the presence of spacing members is e.g. that the position between the first and second sheet members is secured, therewith lowering the risk of undesired repositioning of any of the sheet members upon removal of the backing paper before positioning the device on the skin.

It is to be noted that the first, second and third backing papers are schematically drawn. The arrangement of the said backing papers is discussed ion more detail in FIG. 10.

FIG. 2B shows the assembly of the separate components of FIG. 2A. Once applied to the skin of a patient, the device of FIG. 2 has a two-layered structure, namely the single piece of basic layers, bridging members and spacing members, on the one hand, and the covering sheet on the other.

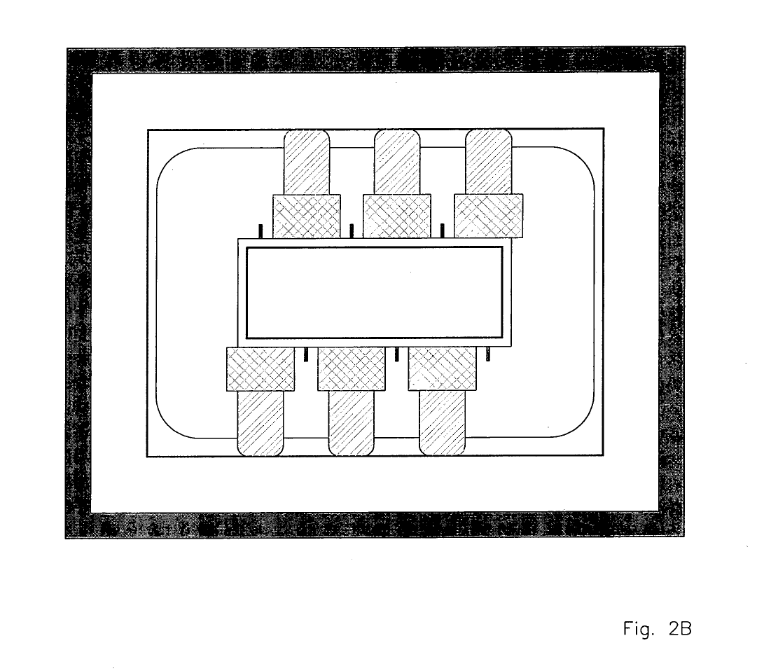

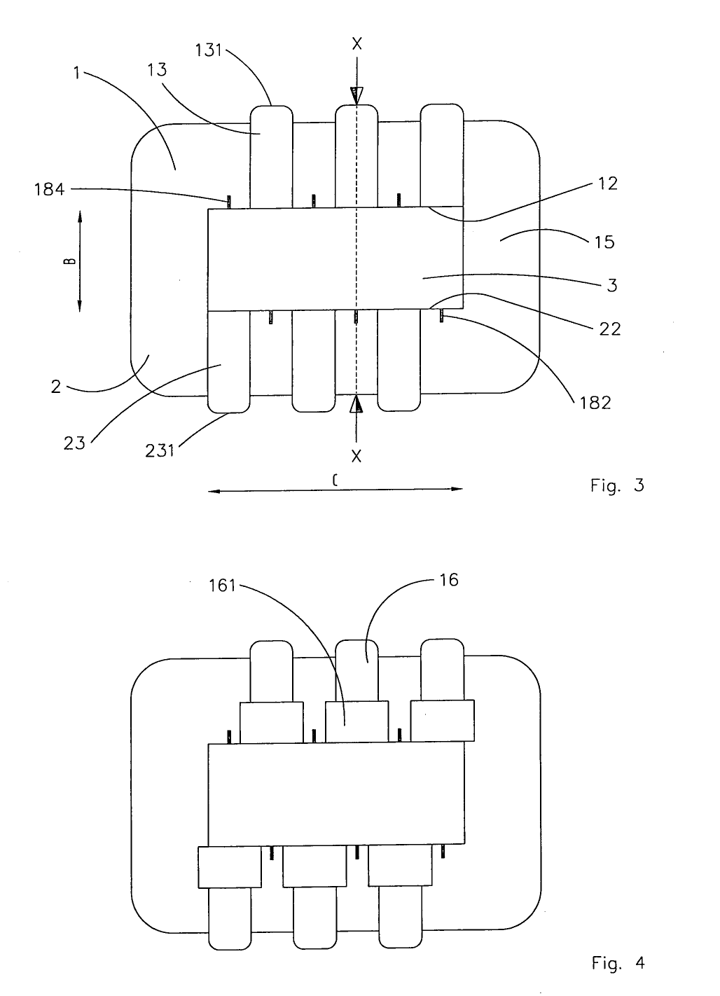

An assembly of first and second sheet members 1, 2, first and second bridging members 13, 23 and spacing members 15 is given in FIG. 3. Also, alignment markers 184 and 182 are shown. Further, the length of the first opposing edge 12 and second opposing edge 22 is indicated with L. Spacing members 15 define a bridging distance B between the opposing edges 12, 22 of sheet members 1 and 2. Bridging members 13 and 23 are folded back over the opposing edges 12 and 22, respectively, both bridging members having a protruding end, or tip, 131, 231, respectively. The area defined by B and L defines the open skin area 3.

In this view, the second surface of the sheet members and spacing members, as well as the first surface of the bridging members face up.

In FIG. 4, the assembly as shown in FIG. 3 is now provided with bridge covering members 16, having a first surface, covering the bridging member there below, and a second surface, facing up. As described in FIG. 1, the bridge covering member can be connected, via the protruding end 131, 231 of the first and second bridging members 13, 23 to the said bridging members. The exceeding end 161′ of the bridge covering member 16 is folded backward at the location of the opposing edges 12, 22 of the sheet members 1, 2 that comprises the said bridging members.

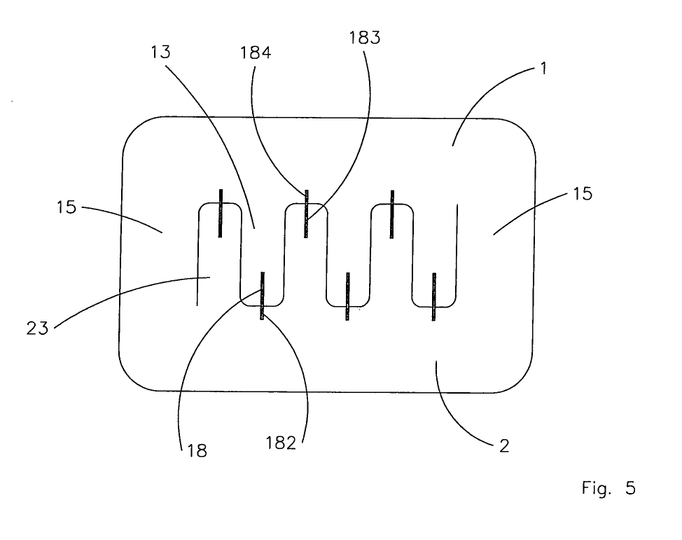

FIG. 5 shows the assembly of FIGS. 3 and 4, now in closed position. The first surface of the first and second sheet members, the first and second spacing members 15 and first and second bridging members 13, 23 are faced up down and comprise an adhesive as described above. Bridging member 13 comprises a first alignment marker 181, cooperating with a second alignment marker 182 on the second sheet member 2, said second alignment marker 182 being located at the second opposing edge 22, opposite to the location of the first bridging member 13. In this unfolded state, the bridging member extends over the open skin area in the direction of the second sheet member, so that the first and second alignment markers can be aligned to one another when the bridging member is adhered to the skin surrounding the wound. Corresponding third and fourth alignment markers 183 and 184 are present on the second bridging member and the first sheet member, respectively.

It is to be noted that FIG. 5 shows the shape of the assembly after production thereof A single sheet of e.g. polyurethane is provided with six lines, forming the first, second, third and fourth alignment markers, each line extending onto a sheet member and a bridging member of the opposing side. Further, the bridging members abut the opposing edge of the opposing sheet member. In this way, the first bridging member extends until the second opposing edge of the second sheet member, and the second bridging member extends until the first opposing edge of the first sheet member.

However, as the sheet material can be stretchable, the bridging members can be stretched upon removing the bridge cover members and pulling the bridging members over the open skin area just before adhering to the skin. By this action, which is further discussed below, the bridging members can be stretched, resulting in extension of the bridging member, so that the protruding ends thereof extend over the opposing sheet members and can be adhered thereon as well. It is however to be noted that a significant portion of the bridging members adhere to the skin, exposed in the open skin area, securing good control of wound closure. By such an extension, the alignment lines 181, 182, and 183, 184, respectively, can be aligned onto one another.

In a very attractive embodiment, as shown in FIGS. 3-5, the bridging members are designed such, that in closed position, i.e. when covering the open skin area in unfolded position, the bridging members lie next to each other and preferably abut one another. More preferably, first and second bridging members abut one another in an alternating fashion. By this arrangement, the complete wound edges will be covered by bridging members, therewith securing an even distribution of the closure forces exerted to the skin, in contrast to the closure by stitching, as discussed above.

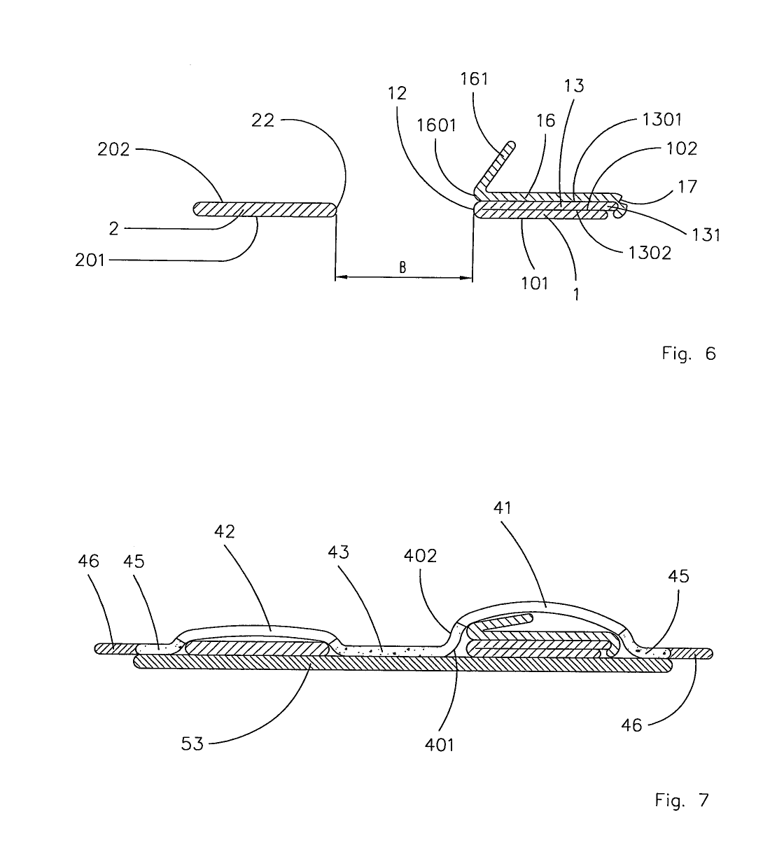

FIG. 6 is a cross section at the line, indicated by arrows X in FIG. 3. It is to be noted that the thickness of the materials indicated are not in scale. At the left, the second sheet member is shown, comprising a first surface 201 and a second surface 202. The first surface 201 comprises an adhesive, for adhering the second sheet member to the skin. Opposed to the second skin member, i.e. at the right in FIG. 6, sheet member 1 is located at a distance B from the second sheet member, distance B defining the bridging distance between the first and second sheet members at their respective opposing edges 12 and 22. The bridging distance B defines the width of open skin area 3. Sheet member 1 is connected to bridging member 13, folded backward upon sheet member 1. Sheet member 1 has a first surface 101, comprising adhesive for adhering to the skin and a second surface 102, opposed thereto. Bridging member 13 has a protruding end 131 and a first surface 1301 and a second surface 1302, facing to the second surface of the sheet member 1. At the protruding end 131 of bridging member 13, a bridge covering member 16 is connected, the bridge covering member having a first surface 1601, covering the adhering first surface 1301 of bridging member 13. The length of the bridge covering member 16 exceeds that of the bridging member 13. The exceeding end 161 of the bridge covering member 16 is folded backward at the location of the opposing edge 12 of sheet member 1 that comprises bridging member 13. The connection between the bridge covering member 16 and the bridging member 13 comprises a breaking line 17, that allows release of the bridge covering member from the bridging member, when the first surface of the bridge covering member is released from the first surface of the bridging member when a pulling force is exerted to the bridge covering member, e.g. by holding the exceeding end 161 of bridging member 16 by hand, exerting a puling force perpendicular to the device, i.e. in upwards direction in the figure to release the bridge covering member from the first surface of the bridging member, followed by a movement in the direction of the opposing second sheet member. Both movements can also performed simultaneously, as will be shown below.

The cross section shown in FIG. 7 corresponds to that of FIG. 6, comprising cover sheet 4 as described for FIG. 2, however without the shielding member being shown. The assembly comprises a sheet of backing paper 53, and a covering sheet 4. The covering sheet 4 has a first surface 401 facing down, and has a first portion 41, covering the first sheet member 1, a second portion 42, covering the second sheet member 2, and a third portion 43, adhering to the backing paper 53, and circumfering portion 45, also adhering to the backing paper. Circumfering portion 45 of the covering sheet 4 comprises and additional edge portion 46. As explained above, regions 41 and 42 are preferably free of adhesive, or, do at least not adhere to the sheet members 1, 2, and the bridging members 13, 23. To that end, a shielding member can be present (not shown).

In another embodiment, a fourth adhesive can be positioned between the second surface of a sheet member and the second surface of a bridging member, back-folded thereon. In that case, the back-folded position of the bridging member is even more secured when the covering sheet is removed. Such a design makes it even possible to allow regions 41 and 42 of the covering sheet 4 to be adhesive as well, as explained above.

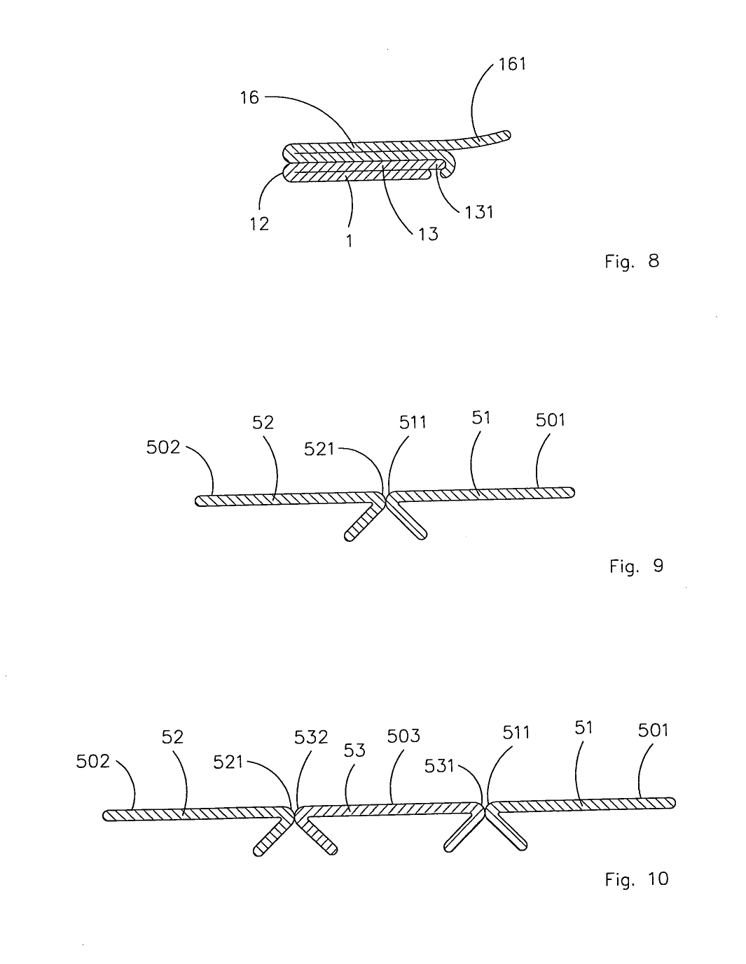

For an even more convenient handling, the bridge covering member has a length, exceeding twice that of the bridging member, so that the end of the bridge covering member 161, extends beyond protruding end 131 of bridging member 13. This is shown in FIG. 8. At the location of the protruding end 131 of bridge member 13, the bridge covering member is folded backward unto bridging member 13, and, at the location of the opposing edge 12 of sheet member 1, the bridge covering member is folded backward again and as it has a length of more than twice of the bridging member, it has a protruding end 161, extending over the protruding end of the bridging member allowing an easy pick-up by hand.

FIG. 9 is a cross section of a first sheet of backing paper 51 and a second sheet of backing paper 52. The first sheet 51 has an edge 511, abutting the second sheet of backing paper at its second abutting edge 521. Both sheets extend beyond their respective abutting edges in order to facilitate manual access to and holding of the sheets. Upper surface 501 of the first sheet of the backing paper and upper surface 502 of the second sheet 52 of backing paper will be in contact with the device according to the present invention, i.e. the first surface of the sheet members and the covering sheet, and in present of the spacing members, which has been explained above. Preferably, these layers are non-adhesive and are hold to the sheet members and the covering sheet by the adhesive surfaces of the said sheet member and covering sheet.

In FIG. 10, the corresponding situation is shown for three sheets of backing paper. Now, the first sheet 51 abuts via its first abutting edge 511, the third sheet (53) of backing paper at the primary third abutting edge (531) thereof Accordingly, the third sheet (53) abuts via its secondary third abutting edge (532). The second sheet adds the second abutting edge 521 thereof Again, all sheets extend beyond there respective abutting edges to facilitate manual handling. Upper surfaces 501, 502 and 503 of the first, second and third sheets, respectively, are to be contacted or are in contact with, the device according to the invention.

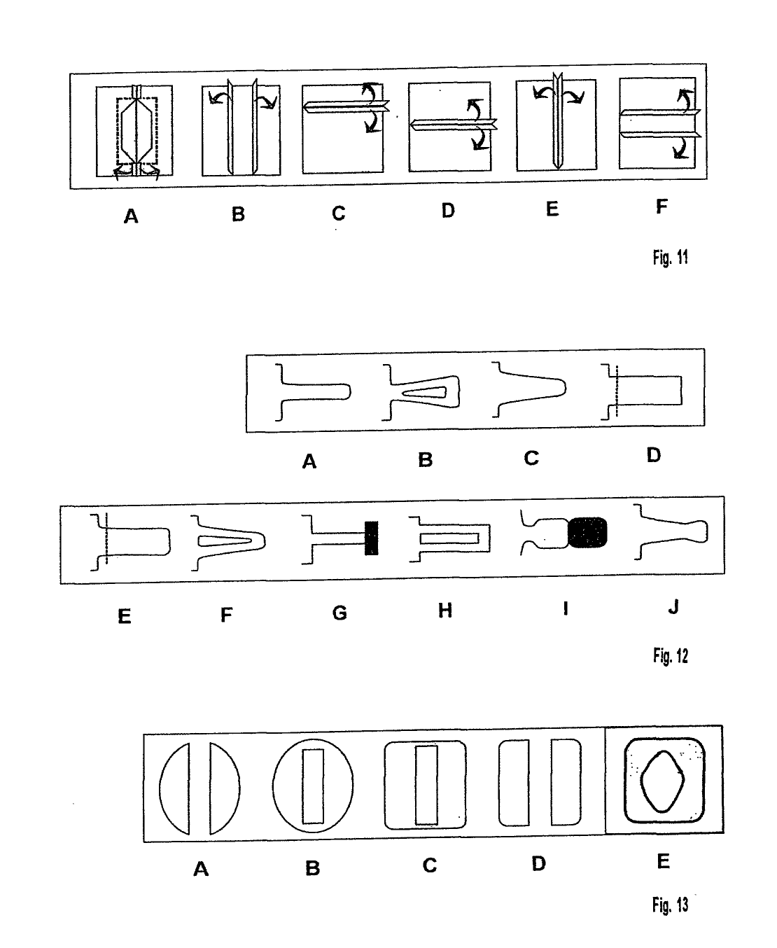

FIG. 11 shows a number of alternative embodiments for the shape of the backing paper. In FIG. 11A, there are two backing papers having abutting edges running in longitudinal direction, i.e. parallel to the opposing edges of the first and second sheet members. As indicated above, multiple sheets of backing material allow proper adherence of the sheet members to the covering sheet therewith avoiding undesired re-positioning of the sheet members upon removal of any of the backing papers. In FIG. 11B, three sheets of backing papers are shown, having longitudinal abutting edges. The embodiment of FIG. 11C comprises two sheets of backing paper having abutting edges, running perpendicular to the sheet members, wherein the upper first sheet member is smaller than the lower second sheet member. In this embodiment, the smaller sheet of backing paper is intended to be removed first and the thus exposed portion of the device is adhered to the skin where after the larger sheet of backing paper is removed as well. In this embodiment, it is preferable that the portion of the device, exposed when the smaller sheet of backing paper is removed, adheres to the covering sheet in order to secure proper positioning of e.g. the sheet members of the device. However, although it is preferable, it is not mandatory. The arrows indicate the direction of removal of the respective backing paper from the assembly.

FIG. 11D corresponds to the embodiment of FIG. 11C, however, there are two sheets of backing paper of identical size.

The embodiment of FIG. 11E corresponds to that of FIG. 11A, although in FIG. 11A, portions of the sheets of backing paper extend more beyond the respective abutting edges than in FIG. 11E.

FIG. 11F corresponds with the preferred embodiment, already explained in FIG. 1.

FIG. 12 shows different embodiments of the shape of bridging members according to FIG. 12E is preferred. However, in particular bridging members The shape according to FIGS. 12A, 12C and 12F can be suitable as well. The bridging members of FIGS. 12G and 121 comprise re-enforced members, suitable to be held by hand in order to facilitate handling and unfolding of the bridging members.

In FIG. 13, several embodiments of the sheet members are given. The embodiments of figures B, C and E comprises pacing members, whereas the embodiments of figures A and D do not. The opposing edges of the first and second sheet members run parallel to one another in the embodiments of figures A-D, but are curved in the embodiment of FIG. 13E. The embodiment of FIG. 13C is preferred.

In FIG. 14, the removal of the bridge covering member, in this case silicone backing paper (however, any suitable material know to the skilled person can be used) is shown. In FIG. 14A, the backing paper is lifted and the contact with the adhering first surface of the bridging member is obviated. Full exposure of the adhering first surface of the bridging member is shown in figure B. The above discussed braking line is seen in FIG. 14C. The second adhesive, present on the first surface of the bridging member, is indicated in grey.

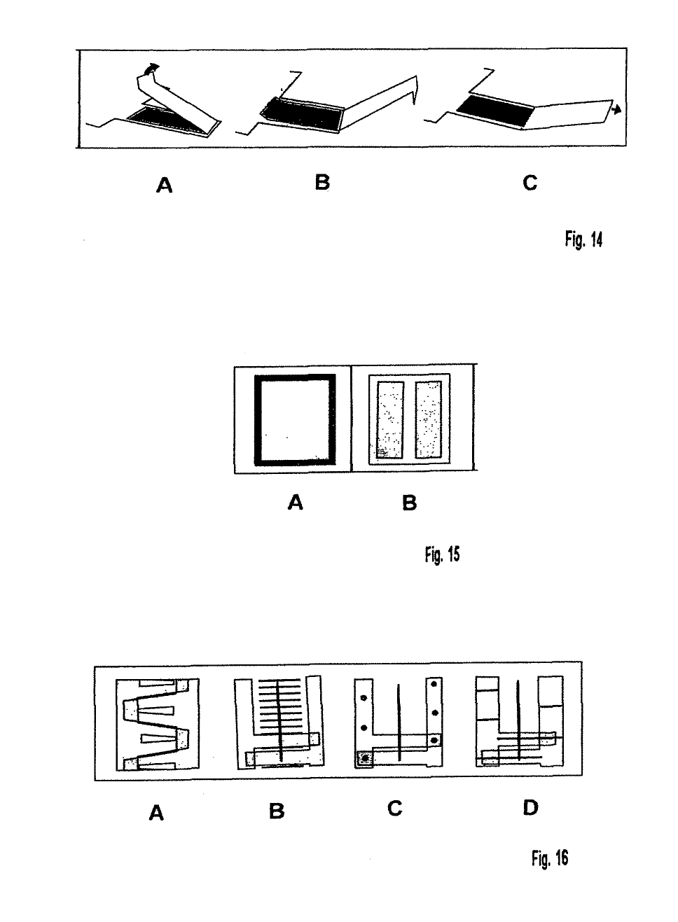

In FIG. 15, two embodiments of covering sheets are shown. In FIG. 1A, the complete first surface of the covering sheet is provided with an adhesive, and the edges thereof are extended with a stronger sheet like material to improve handling of the device and removal of the covering sheet. In FIG. 15B, the grey areas are void of adhesive, as these areas will be contacted with the first and second sheet members and bridging members. As discussed above, it is preferable that the covering sheet does not adhere to these portions of the device.

In FIG. 16, several embodiments of the alignment markers are shown. In figure A, the alignment marker is intrinsically present in the shape of the bridging member. If the bridging members are aligned to one another, a proper positioning of the bridging members is given. In the embodiment of FIG. 16B, the third portion of the covering sheet comprises a marking dye that marks the skin upon contacting the covering member with the skin. The bridging members can be guided along the markings.

The bridging members can, at their protruding ends, comprise a marking element, such as a dot, as shown in FIG. 16C, and a corresponding dot can be present near the opposing edge of the opposing sheet member. The markings can have any shape, and it is also possible that the bridging member has one half of a marking, or as the other half is present on the sheet member. However, this embodiment requires relatively long bridging members which can easily be provided with sheet members according to e.g. FIGS. 13A and 13D, and as explained in FIG. 1. However, in case the sheet members are made of one piece of sheet material, the bridging members have to be stretched to comply with this embodiment.

The embodiment according to FIG. 1D is preferred and comprises alignment lines as discussed in FIGS. 3-5.

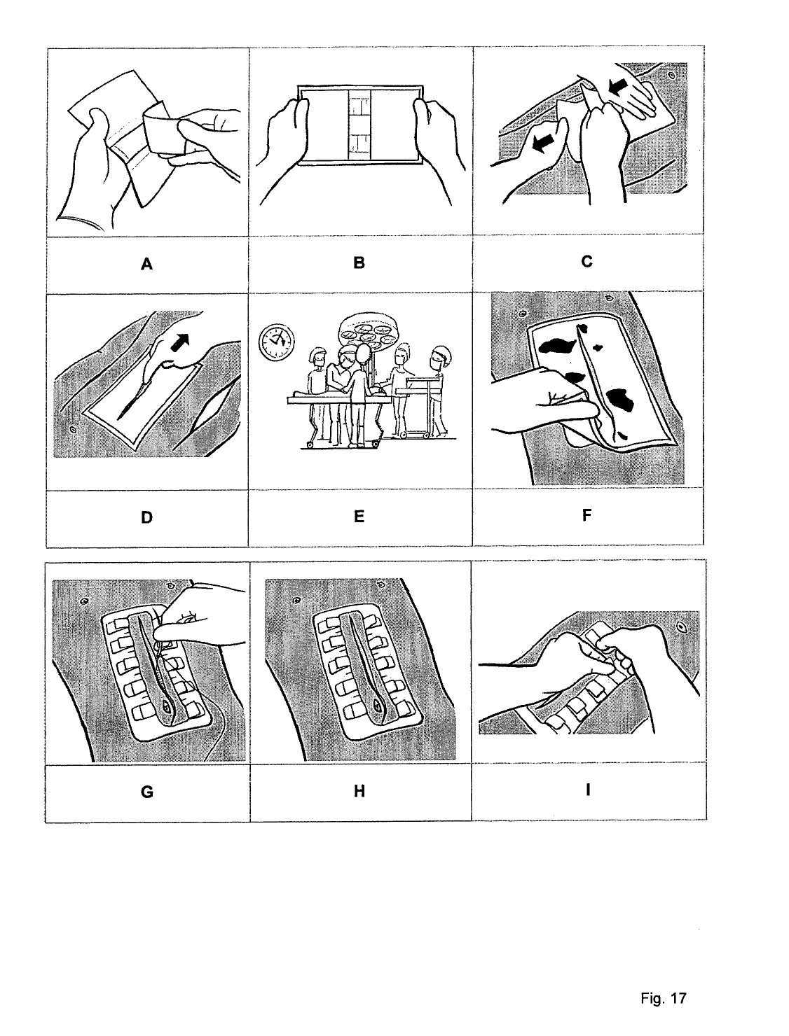

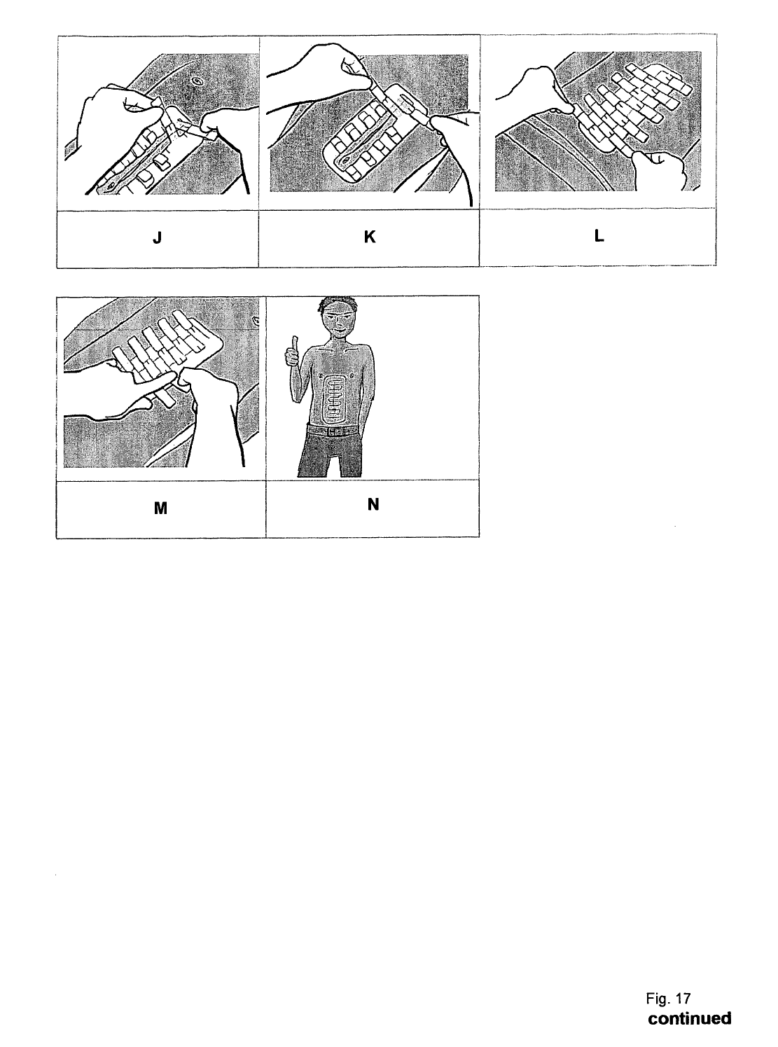

The use of the device is described in FIG. 17. In FIG. 17, the device comprises three sheets of backing paper and is of the type as depicted in FIG. 2. Usually, the device according to the invention is sterilely packed in a suitable bag, or any other suitable packaging means. After removal of the packaging, the surgeon or medical assistant removes the middle (third) part of the backing paper (FIGS. 17A and B) and contacts the exposed portion of the device to the skin of a patient, followed by removal of the first sheet of backing paper, adhering the therewith exposed portion of the device to the skin, and eventually removing the second sheet of backing paper and adhering the therewith exposed portion of the device to the skin, see FIG. 17C. It is to be observed that the patient is preferably human, but the device may also be applicable on animal skin. Then, the surgeon is allowed to make the incision or excision in the skin, in the present case in the abdomen of a human patient. The device is particularly suitable for use on a human, i.e. for human skin. The incision or excision is made through the third portion of the covering sheet, in the open skin area of the device. After the incision is made, the surgeon can operate the patient, see FIG. 17E. It is to be noted that in contract to the device, known from the art, not only incisions, but also excisions can be made using the device of the present invention. With the devices according to the state of the art, excisions can not be made, as closure of the wound occurs through re-abutting of the device layer, still sticking to the wound surrounding. Abutting of these layers will not result in wound closure. After the operation, the covering sheet (also named “incision foil”) is removed, therewith exposing the wound and surrounding skin in the open skin area, shown in FIG. 17G. By removal of the covering sheet, the bridging members are exposed. If necessary a subcutaneous suture can be made. It can be clearly seen that the incision or excision wound is now ready for closure, see FIG. 17H. In FIG. 17I, the exceeding ends of bridge covering members of two adjacent bridging members are hold by hand and pulled upwards, see FIG. 17J, so that the first surface of the bridging member is released from the bridge covering member, which bridge covering member is still connected to the tip of the bridging member. Note the presence of the alignment markers on both the bridging member and the sheet members. The corresponding alignment markers are aligned to one another and the bridging members are adhered to the skin, therewith closing the wound, covered by the respective bridging member. Note that the bridging members lie adjacent to one another, and preferably abut one another, see FIGS. 17K and L. As the bridging members are preferably closed in a pair wise fashion, as shown in FIGS. 17F-L, the device comprises preferably the same number of first and second bridging members, arranged in an alternating fashion.

After positioning of the bridging members, the bridge covering members can be removed, e.g. by exerting additional pulling force, allowing release of the bridge covering member by breaking off from the bridging member, e.g. with the aid of the presence of a breaking line, as discussed above (see FIG. 17M). The device can also be designed such, that a breaking line, as discussed above, is present, which has a tensile strength that is lower than that of the bridging member and of the bridge covering member, so that the bridge covering member breaks from the bridging member before significant deformation of the bridge covering member and/or bridging member can occur. However, some deformation, e.g. stretching as explained above, can be tolerated, as long as the wound closure is secured. The bridge covering members can also be removed during back-folding the bridging members over the wound, i.e. at the moment, depicted in FIG. 17K. The end result of the closed wound is shown in FIG. 17N.

The invention also encompasses devices for closure an incision in the skin of a patient, as are discussed in the introduction, that comprise alignment markers as discussed above. Alignment markers have not yet been used in devices for closing an incision wound, wherein bridging members are used to bridge the wound and adhere to the surrounding skin. Such devices can have all advantages and features as discussed above, but can also comprise a single sheet member, having two portions, which two portions comprises the bridging members, e.g. as described in WO88/08690, herein incorporated by reference. Referring to FIG. 1 thereof, reference number 2 indicates such a sheet member, on which bridging members 6 are mounted. The portion supporting the left bridging member can be regarded as the first sheet member portion, whereas the right bridging member can be regarded as the second sheet member portion. The incision can be made there between. On the other hand, the sheet member portions can be present as separate from one another, e.g. as shown in FIG. 1 showing the above-discussed first embodiment by reference numbers 1 and 2 respectively. However, many variants are encompassed by this embodiment of the invention. Devices that are adhered to the skin of a patient where through the incision is to be made, or devices that are adhered to the skin adjacent to the incision once the incision is made, or before the incision is made, such as of the type e.g. shown in US2004/020470, herein incorporated by reference. Therein, sheet member portions la and lb are separate from one another and each comprise bridging members 5a and 5b, adhering to the opposite sheet member portion. The special feature in this third embodiment of the invention, can be seen in the presence of alignment markers, that help to position the bridging members properly as discussed above.

1. Device for adhering to the skin of a patient, suitable to make a skin incision there through, the device comprising means for closure of the incision wound, characterized in that the device comprises an assembly of:
a first and a second sheet member, each having a first surface and a second surface, the second surface being opposed to the first surface,
the first surface comprising a first adhesive, suitable to adhere the respective sheet member to the skin,
the first and second sheet members being positioned opposite to one another, the first sheet member having a first opposing edge of a first length, the second sheet member having a second opposing edge of a second length, the first and second opposing edges opposing one another, defining a bridging distance there between,
the bridging distance and the first and second length of the fist and second opposing edges of the fist and second sheet members defining an open skin access area to include the wound and surrounding skin therein,
the first and second sheet members comprising, at their first and second opposing edges respectively, at least one bridging member, extending away from the said respective edges and ending in a protruding end, the bridging member comprising a strip of sheet material, having a first surface and a second surface, the first surface being opposed to the second surface, the first surface comprising a second adhesive, suitable to adhere the respective bridging member to the skin, and optionally also to the second surface of the opposing sheet member,
the first and second bridging members being folded backward onto the first and second sheet members respectively, so that at least a portion of the second surface of the bridging member faces the second surface of the respective sheet member,
a covering sheet, comprising a first surface and a second surface 404 the first surface facing the second surface of the first and second sheet members and the open skin area, the covering sheet comprising a first portion, covering the first sheet member, a second portion, covering the second sheet member, and a third portion, covering the open skin area, the first surface of the covering sheet comprising at the third portion thereof, a third adhesive, suitable to be releasably adhered to the skin.

2. Device according to claim 1, wherein the covering sheet comprises a circumfering portion, circumfering the first, second and third portions of the covering sheet, and wherein the first surface of the covering sheet comprises a fourth adhesive at the circumfering portion, which adhesive is suitable to be releasably adhered to the skin.

3. Device according to claim 1, wherein the first surface of the covering sheet covering the first and second sheet members are free of adhesive.

4. Device according to claim 1, wherein the first and second bridging members are an integral part of the first sheet member and the second sheet member respectively.

5. Device according to claim 1, wherein the first bridging member extends until the second opposing edge of the second sheet member, and the second bridging member extends until the first opposing edge of the first sheet member.

6. Device according to claim 1, the assembly further comprising a spacing member, connected to both the first and the second sheet members, defining the bridging distance there between.

7. Device according to claim 6, wherein the spacing member is of sheet material, having a first and second surface, the second surface being opposed to the first, the first comprising an adhesive, suitable for releasably adhering the spacing member to the skin.

8. Device according to claim 6, the assembly comprising two spacing members defining, together with the first and second opposing edges, the open skin area.

9. Device according to claims 6, wherein the first and second sheet members and the spacing member(s) are made of a single piece of sheet material.

10. Device according to claim 1, wherein the covering sheet comprises first visual indication means, indicating the location of the first and second sheet member, and, if present, the location of the spacing member(s).

11. Device according to claim 1, wherein the covering sheet comprises second visual indication means, indicating the location of the open skin access area.

12. Device according to claim 1, wherein the first surface of each of the bridging members are covered by a first surface of a bridging covering member, so as to cover the adhesive first surface of the bridging members, the first surface of the bridge covering members being releasably attached to the first surface of the respective bridging members.

13. Device according to claim 12, wherein the bridge covering member is connected to the protruding end of the respective bridging member, and folded backward, onto the first surface thereof.

14. Device according to claim 13, wherein the connection between the bridge covering member and the bridging member comprises a breaking line, having a tensile strength being lower than that of the bridging member, and of the bridge covering member, allowing release of the bridge covering member from the bridging member by exerting a pulling force on the bridge covering member.

15. Device according to claim 14, wherein the length of the bridge covering member exceeds that of the respective bridging member, the exceeding end of the bridge covering member being folded backward at the location of the opposing edge of the sheet member that comprises the said bridging member.

16. Device according to claim 15, wherein the length of the bridge covering member exceeds twice that of the respective bridging member, so that the exceeding end of the bridge covering member extends beyond the end of the bridging member.

17. Device to claim 1, wherein the first bridging member comprises a first alignment marker at the protruding end thereof, and the second sheet member comprises a second alignment marker at the second opposing edge, opposite to the location of the first bridging member, so that, upon unfolding the bridging member over the open skin area in the direction of the second sheet member, the first and second alignment markers can be aligned to one another.

18. Device according to claim 17, wherein the second bridging member comprises a third alignment marker at the protruding end thereof, and the first sheet member comprises a fourth alignment marker at the first opposing edge, opposite to the location of the second bridging member, so that, upon unfolding the bridging member over the open skin area in the direction of the first sheet member, the third and fourth alignment markers can be aligned to one another.

19. Device according to claim 1, the assembly further comprising at least one sheet of backing material releasably attached to the first surface of the first and second sheet members, as well as to the third portion of the covering sheet, covering the open skin area.

20. Device according to claim 1, the assembly further comprising a first and a second sheet of backing material, the first sheet and second sheet abutting one another via a first abutting edge and a second abutting edge of the first and second sheet of backing paper respectively.

21. Device according to claim 1, the assembly further comprising a first, a second and a third sheet of backing material, the third sheet comprising a primary abutting edge and a secondary abutting edge, opposed to one another, the third sheet and the first sheet abutting one another via a first abutting edge of the first sheet and the primary abutting edge of the third sheet, the third sheet and the second sheet abutting one another via a second abutting edge of the second sheet and the secondary abutting edge of the third sheet.

22. Device according to claim 20, wherein the first and second abutting edges are perpendicular to the first and second opposing edges of the first and second sheet members.

23. Device according to claim 20, wherein the first and second abutting edges cross the open skin area.

24. Device according to claim 1, wherein the sheet material of the first and second sheet members comprise polyurethane foil.

25. Device according to claim 1, wherein the covering sheet comprises polyurethane foil.

26. Device according to claim 1, wherein the thickness of the sheet of the first and second sheet members is between 20 and 60 μm, more preferably between 25-50 μm, most preferably about 35 μm.

27. Device according to claim 1, wherein the first, second, third and fourth adhesives is a hotmelt adhesive based on polyolefine.

28. Use of a device according to claim 1 for allowing an incision or excision wound to be made in skin through the said device, and for subsequent closing the said wound, comprising the steps of:
a) contacting the first surfaces of the first and second sheet members and the edges of the covering sheet and the third portion of the covering sheet with the skin such, that the said third portion of the covering sheet contacts the skin on the location where the incision is to be made, and adhering the device to the skin,
b) allowing the incision or excision to be made by cutting through the third portion of the covering sheet,
c) removing the covering sheet, therewith exposing the wound and the surrounding skin in the open skin area,
d) folding back the first bridging member(s) in the direction of the opposite opposing edge of the second sheet member and adhering the said first bridging member(s) to the skin in the open skin area, therewith at least partially closing the wound,
e) folding back the second bridging member(s) in the direction of the opposite opposing edge of the first sheet member and adhering the said second bridging member(s) to the skin in the open skin area, therewith at least partially closing the wound.

29. Use according to claim 28, wherein steps d) and e) are performed simultaneously.

30. Use according to claim 28 of a device according to any of the claims 17-27, wherein step a) comprises removal of at least one backing paper from the device before contacting an adhering the therewith exposed portion of the device to the skin.

31. Use according to claim 30, wherein step a) comprises removal of the third backing paper, contacting an adhering the therewith exposed portion of the device to the skin, removing the first backing paper, and adhering the therewith exposed portion of the device to the skin, and removing the second backing paper, contacting an adhering the therewith exposed portion of the device to the skin.

32. Use according to claim 29 of a device according to any of the claims 10-16, wherein step d) comprises releasing the first surface of the bridge covering member from the first surface of the bridging member before adhering the bridging member to the skin in the open skin area.

33. Use according to claim 29 of a device according to claim 17 or 18, wherein in step d) the bridging member is adhered to the skin such, that the first and second, and, if present, the third and fourth alignment markers, are aligned to one another respectively.

34. Device for closure of an incision in the skin of a patient, characterized in that the device comprises:
a sheet member having two opposite portions, each sheet member portion having a first surface and a second surface, the second surface being opposed to the first surface,
the first surface comprising a first adhesive, suitable to adhere the respective sheet member portion to the skin,
the first and second sheet member portions being positioned opposite to one another, allowing the incision to be made or to be present there between,
the first and second sheet member portions each comprising at least one bridging member, each bridging member comprising a strip of sheet material ending in a protruding end, each bridging member having a first surface and a second surface, the first surface being opposed to the second surface, the first surface comprising a second adhesive, suitable to adhere the respective bridging member to the skin and/or to the second surface of the opposing sheet member portion,
the first bridging member comprising a first alignment marker at the protruding end thereof, and the second sheet member portion comprising a second alignment marker, opposite to the location of the first bridging member, so that, upon adhering the bridging member to the opposing second sheet member portion, or to the skin while abutting against the said second sheet member portion, the opposing first and second alignment markers can be aligned to one another.

35. Device according to claim 34, wherein the second bridging member portion comprises a third alignment marker at the protruding end thereof, and the first sheet member portion comprises a fourth alignment marker, opposite to the location of the second bridging member, so that, upon adhering the bridging member to the opposing first sheet member, or to the skin while abutting against the said second sheet member portion, the third and fourth alignment markers can be aligned to one another.

36. Device according to claim 34, wherein the first and second bridging members being folded backward onto the first and second sheet member portions respectively, so that at least a portion of the second surface of the bridging member faces the second surface of the respective sheet member portion.

37. Device according to claim 34, wherein the first and second sheet member portions are present as separate from one another.
